(19) United States
(12) Patent Application Publication
(10) Pub. No.: US 2013/0126760 A1
(43) Pub. Date: May 23, 2013

(54) UV LIGHT SYSTEM WITH SATELLITE UV UNITS

(71) Applicant: TSK Products LLC, Eatontown, NJ (US)

(72) Inventors: Eric S. Klein, New York, NY (US); Michael A. Klein, Eatontown, NJ (US)

(73) Assignee: TSK Products LLC, Eatontown, NJ (US)

(21) Appl. No.: 13/678,687

(22) Filed: Nov. 16, 2012

Related U.S. Application Data
(60) Provisional application No. 61/563,165, filed on Nov. 23, 2011.

(51) Int. Cl. A61L 2/10

(52) U.S. Cl. CPC A61L 2/10
USPC 250/492.1

(57) ABSTRACT

A UV sanitizing system with satellite UV units has a wheeled base UV sterilization unit and at least one satellite UV sterilization unit. All units are capable of generating UV-C from multiple locations within a room or other enclosed space. A timer regulates the anti-bactericidal dose of UV-C administered to the area. Alternatively, sensors may be used to measure the UV energy received by an area within a space and to provide data to a controller. The controller may determine from the sensor data when the area within the location has received sufficient UV energy and control the illumination sources to reduce the UV exposure of the area. When not in use, the satellite UV units can be stored by attaching them to a receiving slot in the wheeled base portion of the UV sanitizing system.

### CROSS REFERENCE TO RELATED APPLICATIONS

This application claims priority under 35 U.S.C. §119(e) from provisional application No. 61/563,165 filed Nov. 23, 2011. The 61/563,165 provisional application is incorporated by reference herein, in its entirety, for all purposes.

### BACKGROUND

Various embodiments illustrated herein relate to devices and methods for sanitizing surfaces within a room using embodiments of a UV light system with satellite UV units that use ultraviolet (UV) light at bactericidal frequencies for the sanitizing of room surfaces in order to inhibit the spread of nosocomial infections in hospitals and other patient care facilities.

Nosocomial, or hospital acquired, infections are common, costly, and sometimes lethal. A recent review of such infections in the cardiac surgery unit of a major hospital revealed a nosocomial infection rate of 27.3% that more than doubled the mortality rate for afflicted patients. The nature of bacteria acquired in the hospital setting differs significantly from bacteria found in a community setting, primarily in their resistance to antibiotic therapy. Historically, staphylococci, pseudomonads, and Escherichia coli have been the nosocomial infection troika; nosocomial pneumonia, surgical wound infections, and vascular access-related bacteremia have caused the most illness and death in hospitalized patients; and intensive care units have been the epicenters of antibiotic resistance. Acquired antimicrobial resistance is the major problem, and vancomycin-resistant Staphylococcus aureus is the pathogen of greatest concern. The shift to outpatient care is leaving the most vulnerable patients in hospitals. Aging of our population and increasingly aggressive medical and surgical interventions, including implanted foreign bodies, organ transplantations, and xenotransplantation, create a cohort of particularly susceptible persons. Renovation of aging hospitals increases the risk of airborne fungal and other infections.

Significant morbidity, mortality, and costs are associated with these infections. Many factors contribute to these dangerous infections. Most notably is the overuse of antibiotics and poor personal hygiene, such as hand washing. Abundant evidence exists, however, that the hospital environment itself contributes to the problem by harboring virulent strains of bacteria, fungi, and viruses, and that many methods commonly used are ineffective and may actually spread contaminants.

Attempts to eradicate surface contaminates from the hospital setting have varied greatly in strategy and success. These have ranged from antiseptic soaps to fumigation with formaldehyde gas. Topical antiseptics are problematic for several reasons. First, they have recently been shown to actually induce antibiotic resistances, and thus may be adding to the problem. Secondly, many surfaces, such as keyboards, television sets, and monitoring controls, are difficult if not impossible to decontaminate with liquid disinfectants without harming the electronics. Gas disinfection, while effective, is time consuming, hazardous to workers, and environmentally unwise.

Ultraviolet (UV) light has been long used for disinfection and sterilization. Ultraviolet light may be produced artificially by electric arc lamps. Recently, the widespread availability of low to medium pressure mercury bulbs has led to the development of devices that use UV-C to decontaminate water supplies. UV-C is a high frequency wavelength of light within the ultraviolet band, and has been shown to be the most bactericidal type of ultraviolet light. UV-C has wavelengths of about 2800 Angstroms to 150 Angstroms. To date, there are no published efforts to use UV-C to decontaminate or disinfect larger areas, such as operating rooms. The only recent availability of the appropriate bulbs, as well as significant safety concerns regarding worker exposure to UV-C, likely contribute to the lack of efforts to use UV-C outside of self-contained water purification systems.

### SUMMARY

In an embodiment a UV light system with satellite UV units includes UV-C generators, such as mercury bulbs, which generate UV-C from multiple locations within a room or other enclosed space. By way of illustration and not by way of limitation, the enclosed space may be in a hospital, a doctor's office, or in a clinic. The enclosed space may include a patient ward, an operating room, an emergency room, an intensive care unit, a blood donation facility, and a kidney dialysis facility.

The device has a timer to regulate the anti-bactericidal dose of UV-C administered to the area. Once the set time has elapsed, the unit automatically shuts down. Alternatively, fixed or portable/removable sensors can sense the amount of UV-C delivered and send a signal to the base unit or to satellite units to shut down when an antibacterial dose of UV-C has been delivered. When not in use, the satellite UV units can be stored by attaching them to a base portion of the UV light system. Alternatively, the satellite units are self supporting/standing and can be associated with base units as more fully described herein.

These and other features of the present invention will become readily apparent upon further review of the following specification and drawings.

### DESCRIPTION OF THE DRAWINGS

FIG. 1 illustrates a perspective view of a UV light system with satellite UV units according to an embodiment, shown with the satellite units attached to the base unit.

FIG. 2 illustrates a perspective view of the UV light system of FIG. 1, shown with a pair of the satellite units extended from the base unit.

FIG. 3 illustrates a perspective view of an alternative embodiment of a UV light system with satellite UV units according to an embodiment, the system having a single satellite unit shown extended from the base unit.

FIG. 4 illustrates a block diagram of a control circuit for a UV light system with satellite units according to an embodiment.

FIG. 5 illustrates an embodiment having ceiling illumination from a satellite unit and wherein all satellite units receive wired or wireless signals from the base unit.

FIG. 6 illustrates an embodiment wherein sensors sense UV-C energy and provide signals to the base unit.

FIG. 7 illustrates an embodiment wherein a central control facility manages the operations of multiple base units.

FIG. 8 illustrates a reflector embodiment for UV Sanitizing

FIG. 9 illustrates UV-C irradiation of a series of patent furniture, each having associated UV-C sources, with control of the UV-C sources by a based station and/or a central control facility.

Similar reference characters denote corresponding features consistently throughout the attached drawings where appropriate.

### DETAILED DESCRIPTION

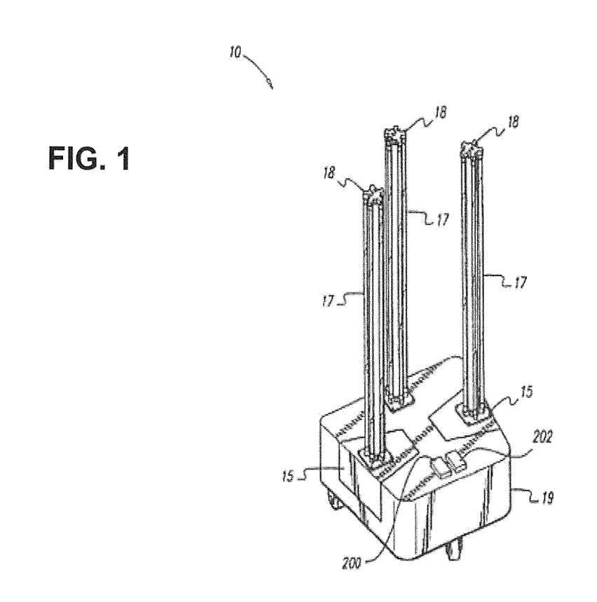

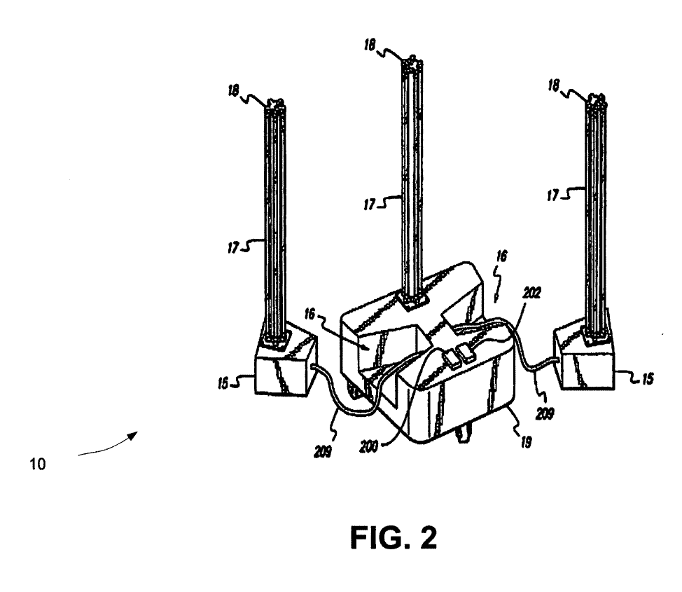

As shown in FIGS. 1 and 2, in a first embodiment the UV light system 10 with satellite UV units has a plurality of UV-C emitter assemblies 17, which may include, e.g., high intensity mercury vapor quartz bulbs, which generate sterilizing UV-C at multiple locations by placement of a wheeled, transportable base unit 19 and satellite units 15 at separate locations within the room or other enclosed space. The wheeled base unit 19 has at least one slot 16 that is of the same shape and slightly larger in dimension than peripheral sidewalls of a satellite unit 15 so that the satellite unit 15 can fit in the slot 16 for easy transport of the wheeled base 19 and the satellite units 15 to the desired sterilization room or space and to facilitate storage of the satellite units 15.

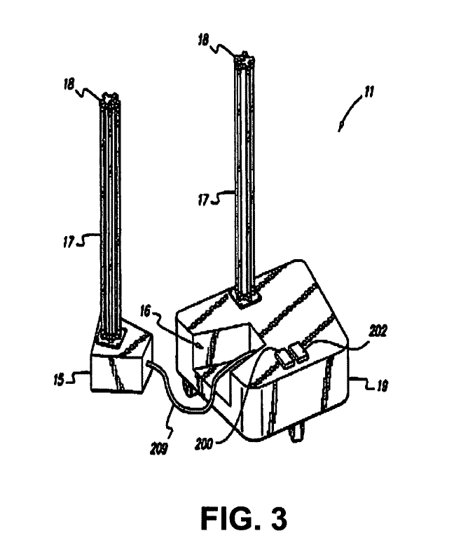

Alternatively, as shown in FIG. 3, a system 11 may be provided with a base unit 19 having only one slot 16 to accommodate a single satellite unit 15. The UV-C light assemblies 17 extend vertically from the wheeled base unit 19 and from satellite base units 15 and are terminated at the top by a cap 18. The satellite units 15 and base unit 19 can be located at two or more locations in a room to be treated.

Referring to FIGS. 1, 2 and 3, the base unit 19 has an escape timer 200 that allows personnel to leave the treatment location before activation of the UV light system. The base unit 19 may also have an operational timer 202 to regulate the anti-bactericidal dose of UV-C administered to the location. The base unit and/or the satellite units may also be equipped with wheels to facilitate transport of the base unit and the satellite units. In an alternate embodiment the base unit and/or the satellite units may not be equipped with wheels.

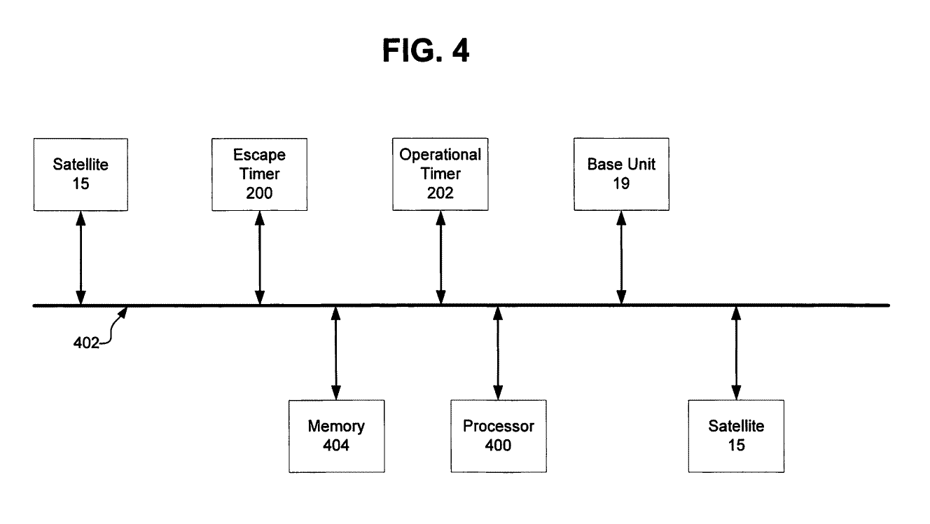

Referring to FIG. 4, a block diagram of a control circuit for a UV light system with satellite units is shown. The wheeled base unit 19 has control electronic circuitry, including an escape timer 200, an operational timer 202, a processor 400 and a memory 404. The escape timer 200 is set to allow personnel to leave the treatment area before activation of the system 10. The operational timer 202 may be used to control satellite units 15 based on a pre-determined amount of time.

In an embodiment, software instructions are stored in the memory 404. The software instructions are accessible by the processor 400. When executed by the processor, the software instructions cause the processor to perform operations described in various embodiments (below). The processor and the memory are connected to the base unit 19 and to the satellite units 15 via a control bus 402.

The control bus 402 may be either wired or wireless, for wired or wireless control of satellite units 15. Further, the base unit may receive actuation signals from outside the room via a wireless control or via signals being sent to the base unit 19 from a central control facility, as more fully explained below.

In an embodiment, a cable 209 connects electronic circuitry of the base unit 19 to electronic circuitry of the satellite unit 15. In another embodiment, control from the base unit 19 to the satellite unit(s) 15 may be effected by Bluetooth or by any other suitable wireless technology. Instructions for operating the satellite units may be transmitted in a wireless mode from base unit 19 to each of the satellite units 15. In this manner, any number of satellite units 15 may be advantageously placed in various areas in the room to be sanitized.

The satellite units 15 may receive power from self-contained batteries or from the main electrical system of the enclosed space or room. In another embodiment, wireless satellite units 15 may be either fixed in designated places of the enclosed space or be transportable in the same manner as the wired units.

In an alternate embodiment, any number of satellite units 15 may be registered with a shared base unit 19 and controlled by the base unit 19. When a satellite unit 15 is desired to be used together with the base unit 19, it is brought within range of base unit 19 and a registration process, known in the art, takes place allowing the new satellite unit 15 to be registered and controlled by base unit 19. In this manner, an inventory of satellite units 15 may be obtained and advantageously used with any of a number of different base units 19 without having to be dedicated to a specific base unit.

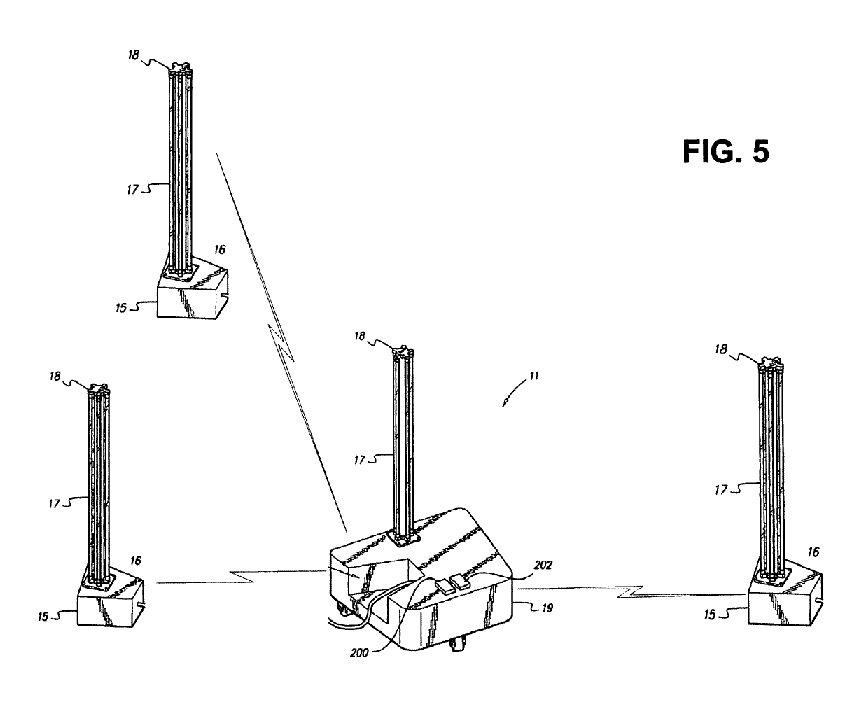

Referring now to FIG. 5 an alternate room embodiment is illustrated. In this embodiment, satellite unit 15 may be placed in various locations in the room and/or in the ceiling of the room to be sanitized. The satellite units can operate from normal power that is generally given to normal ceiling fixtures to be sanitized or may operate on rechargeable batteries so that they can be placed and removed from a particular location in the ceiling of a room to be sanitized. These satellite units, while operating in a fixed location in the ceiling of a room to be sanitized, are registered with the base unit 19 when the base unit 19 is moved into place in the room to be sanitized. Thus multiple base units 19 may be alternatively used in the same room and simply go through a registration process with the in-place satellite units 15 already in the room in fixed locations.

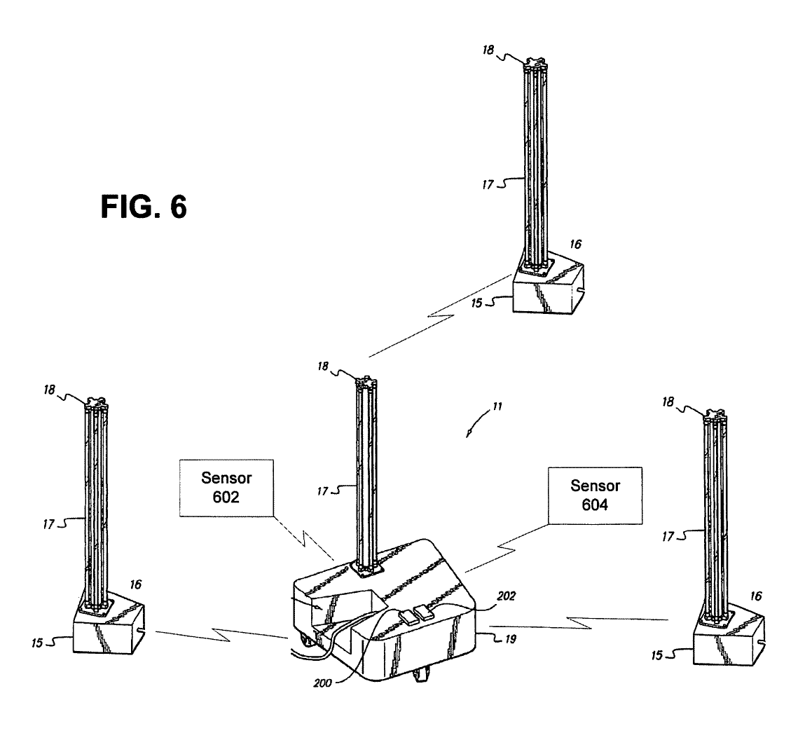

Referring now to FIG. 6, yet another embodiment is illustrated. In another alternate embodiment, transportable wired or wireless UV sensors 602, 604 are placed in the room to be sanitized in various locations. UV sensors 602, 604 then sense the amount of UV that is striking the surfaces in the areas of the sensors and provide sensor data to base unit 19. The UV sensor data is evaluated by the processor 202 to determine whether an appropriate amount of UV energy has been received in a particular area. When processor 202 (also illustrated as element 400 in FIG. 4) determines that an appropriate amount of UV energy has been received, the processor 202 may signal one or more satellite units 15 that are providing the illumination to a particular area to power down. Thus, an appropriate dosage can be delivered to any particular area of the room without having to have all satellite units remain on for a fixed period of time.

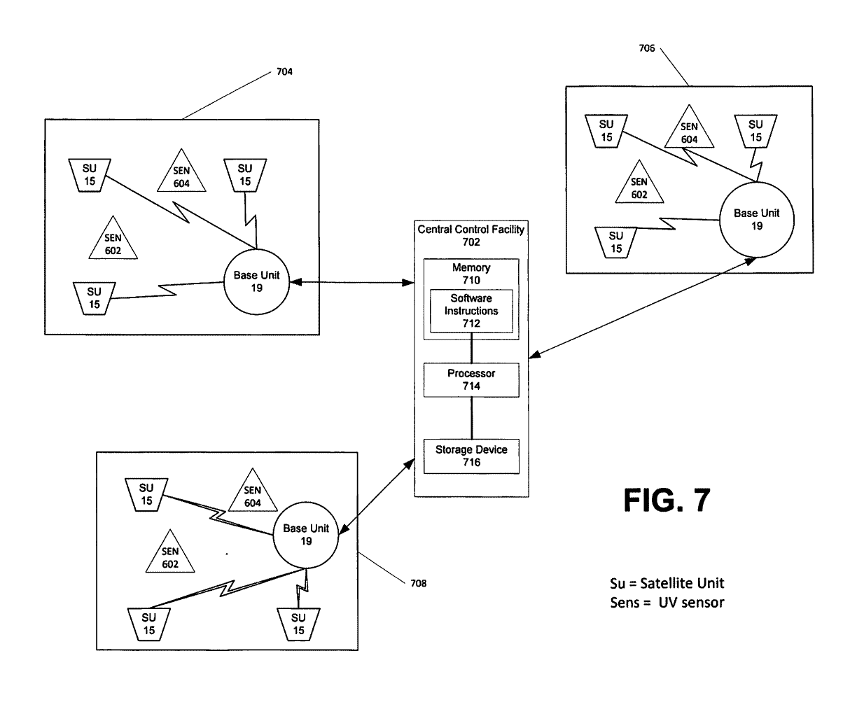

Referring now to FIG. 7, locations 704, 706 and 708 each have a sanitizing module that includes a base unit 19, associated satellite units 15 and sensors 602 and 604. The satellite units 15 may be controlled locally by the base unit 19 or, in the alternative, base units 19 may send signals to a central control facility 702. For example, the central control facility may be located in a hospital or in a facility remote from the hospital, such that the sanitization of a series of rooms can be centrally managed from a central location. In an embodiment, the central control facility 702 includes a memory 710 that is accessible to a processor 714. Software instructions 712 are stored in the memory 710 and may be executed by the processor 714. The central control facility under control of the processor 714 may control the start and stop times of base units 19 or may instruct the base units 19 when to turn on or turn off individual satellite units 15. Additionally, the central control facility 702 may receive sensor data from each base unit 19. Each sensor provides UV energy monitoring information to the base unit 19 and each base unit 19 in turn provides UV illumination information to the central control location 702, so that satellite units and base stations can be turned on and off as needed.

The software instructions 712 stored in the memory 710 may further enable the central control facility to maintain a record of when a particular space has been sanitized. The record, which may be stored in storage device 716, may include the date and time that the sanitizing operation was started and completed, and the sensor data indicating the amount of UV that was delivered to areas within the space.

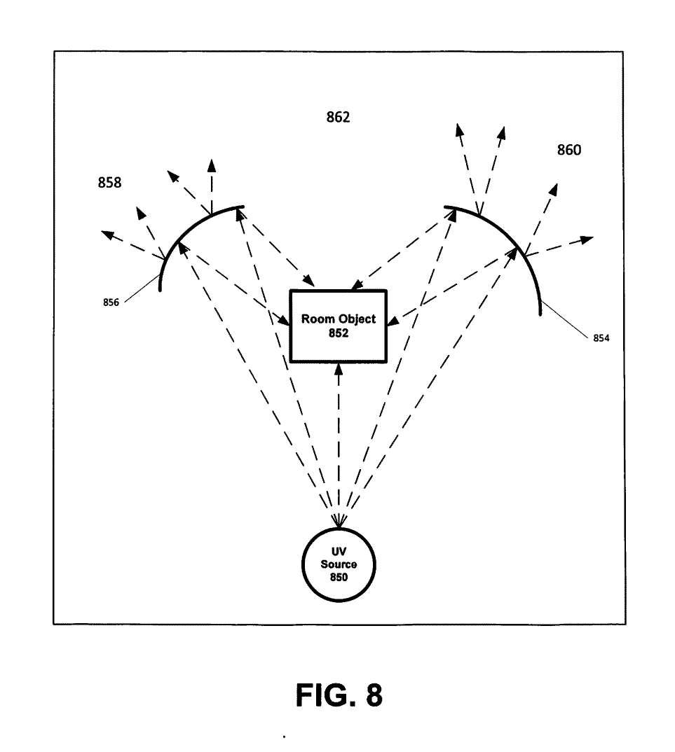

Referring now to FIG. 8, in yet another embodiment of the room sanitizer, UV source 850 emits UV energy (shown in dashed lines in FIG. 8) that will reach the surface of a room object 852 such as a bed or other physical object, and sanitize the surface facing the UV source 850 by direct radiation. Additionally, in this embodiment separate UV reflectors 858 and 860 are placed advantageously surrounding the room object 852 so that reflected energy from UV source 850 is reflected from reflectors 858 and 860 thereby allowing the sides and back of room object 852 to also receive reflected UV energy in the manner shown by the dashed lines. Reflectors 858 and 860 may be solid reflectors, reflecting substantial amounts of UV energy reaching them.

In yet another embodiment also illustrated in FIG. 8, the separate UV reflectors 858 and 860 may also comprise a perforated reflector material. The perforations in separate UV reflectors 858 and 860 permit UV radiation reaching those reflectors to pass through the perforations thereby allowing an advantageous amount of UV energy to pass through the reflectors, illustrated as 858 and 860, and reach other portions of the room 862, thereby avoiding problems when shadows are cast by objects within the room. However, the remaining surfaces of reflectors 858 and 860 continue to reflect UV energy to irradiate the sides and back of room object 852. The reflectors 858 and 860 would be perforated, vertical, reflectors having a parabolic cross section. The perforations will help minimize the shadow behind the dish. The perforations would flare out in the direction of the original light path, spreading the light out to cover any shadow created by the reflectors.

It should also be noted that the positioning of the separate UV reflectors in FIG. 8 is for illustrative purposes only. For example it may only be required to have a single reflector for a particular situation. Alternatively there might be additional UV sources together with one or more reflectors advantageously placed in the room to ensure that appropriate amounts of sanitizing UV energy are delivered.

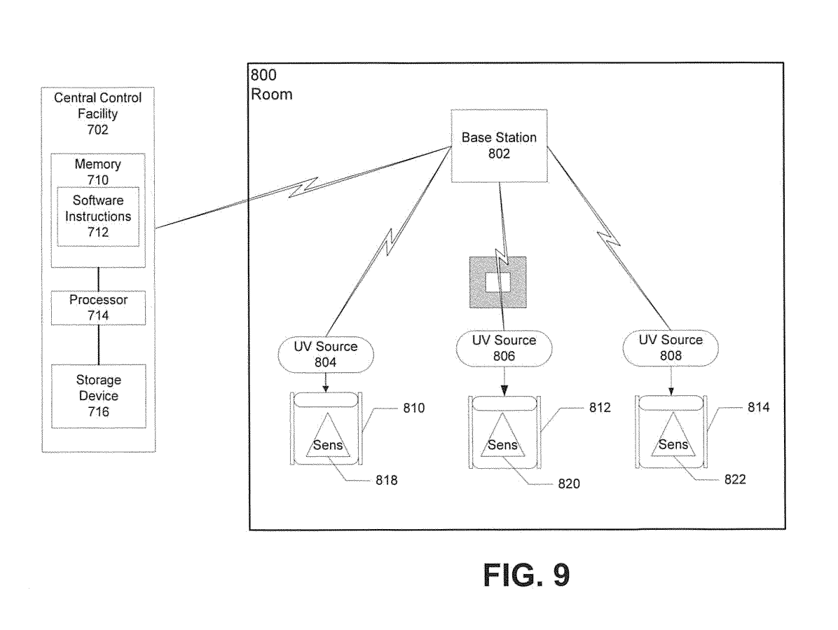

Referring now to FIG. 9, an embodiment of a room having multiple similar patient locations is illustrated. Using various embodiments described herein, UV illumination can be provided to various types of fixed locations. For example, in a dialysis situation, where multiple chairs may be in a particular location 800, UV illumination can be advantageously provided through a combination of ceiling fixtures and movable satellite units 804, 806, 808. In an alternative embodiment, ultraviolet illumination may be flexibly located in, for example, a dialysis chair 810, 812, 814 such that when one patient has completed dialysis, its UV lamp 804, 806, 808 can be moved into place from a fixed location in the chair or nearby such that the UV illumination can be provided to the arms of the chair, and other seating areas in order to prevent germs being transmitted to the next dialysis patient. In this embodiment, the UV lamp can be controlled either by a timer or base station 802 having a previously established efficacious timing cycle or may be regulated by a transportable, or seat specific UV sensor 818, 820, 822 that will send a “turnoff” signal to a control unit for the particular chair, or to the base station 802 when a particular chair has received sufficient ultraviolet illumination. Thus in a situation where a room has a plurality of dialysis chairs, or blood transfusion chairs/beds or any other type of furniture, each individual piece of furniture and its associated ultraviolet illumination can be controlled locally via a sensor and local control unit, by a base unit 802 receiving signals from seat-associated sensors, or a central control facility 702 to which all ultraviolet dosage signals may be sent and control signals sent back to each piece of patient furniture.

In any of the embodiments noted herein, the UV source(s) could be pulse UV or steady state UV sources. Both the pulse UV source and the steady state UV source may be selected depending on the physical situation, to generate advantageous amounts of UV energy for sanitizing rooms and surfaces within the room.

The various embodiments expressed herein are not meant to be limiting. For example chairs have been discussed as being subject to UV illumination; individual patient beds are also within the scope of the various embodiments disclosed herein. Each patient bed would have its own integral ultraviolet illumination source providing sanitizing capability. Each source could be locally timed at each bed with a control unit thereby allowing UV illumination to be delivered over a specific period of time. Additionally, each piece of patient furniture could be connected in a wired or wireless manner to a base station located in the room or nearby with control of illumination being effected by the base station.

Each piece of patient furniture can have integral UV sensors that allow for an appropriate amount of UV illumination to be received. When an appropriate threshold pre-established sanitizing level is received by the sensor, sensors can send a signal to the illumination source to turn off the UV illumination. Alternatively, the sensors may send a signal to the base station or to a central control facility each of which could send a signal to the associated UV source to turn off when the appropriate amount of energy has been delivered.

It should also be noted that all references to communications and exchanging of instructions and signals among the sensors, base stations, and any central control facility, may be either by wired or wireless communication. The figures associated with the various embodiments disclosed herein are not meant to be limiting in terms of the communication methodology being used.

In an alternate embodiment, the base unit is also controlled via a wireless system from a handheld or portable controller or from signals sent from a central control facility.

It is to be understood that the embodiments illustrated herein are not meant to be limiting but provide illustrations of that which is considered by the inventors to be within the scope of the following claims.

What is claimed is:

1. A UV sanitizing system comprising:
one or more UV sensors;
one or more satellite assemblies each comprising a satellite UV illumination source;
a base unit in communication with the one or more UV sensors and the one or more satellite assemblies, the base unit comprising:
one or more base UV illumination sources;
a memory having stored therein software instructions;
a processor in communication with the memory and configured to receive software instructions that when executed by the processor cause the base unit to perform operations comprising:
delivering UV illumination to a location in which the one or more satellite assemblies and the base station are present;
receiving sensor data from the at least one UV sensor, wherein the sensor data is indicative of the amount of UV energy received by the UV sensor;
determining from the sensor data when an area within the location has received sufficient UV energy; and
controlling at least one of the UV illumination sources of the one or more satellite assemblies and the one or more UV illumination sources of the base station to reduce the UV exposure of the area within the location when the area within the location has received sufficient UV energy according to the sensor data.

2. The UV sanitizing system of claim 1, wherein the communication between the base unit and the one or more satellite assemblies is over a link selected from the group consisting of a wired connection and a wireless connection.

3. The UV sanitizing system of claim 1, wherein the UV sensor is selected from the group consisting of a portable sensor and a sensor having a fixed location.

4. The UV sanitizing system of claim 3, wherein the fixed location is selected from the group consisting of a fixed location in a room to be sanitized and a piece of patient furniture.

5. The UV sanitizing system of claim 4, wherein the patient furniture is selected from the group consisting of chairs, beds, and operating tables.

6. The UV sanitizing system of claim 1, wherein controlling the at least one of the UV illumination sources of the one or more satellite assemblies and the one or more UV illumination sources of the base station to reduce the UV exposure of the area within the location comprises terminating UV illumination from a selected satellite UV illumination unit when a pre-determined amount of UV illumination is received by the sensor.

7. The UV sanitizing system of claim 1, wherein the location is selected from the group consisting of a doctor's office, a clinic, a patient ward, an operating room, an emergency room, an intensive care unit, a blood donation facility, and a kidney dialysis facility.

8. The UV sanitizing system of claim 1 further comprising a separate UV reflector.

9. The UV sanitizing system of claim 8, wherein the UV separate reflector comprises multiple separate UV reflectors.

10. The UV sanitizing system of claim 8 wherein the separate UV reflector comprises a perforated UV reflector.

11. The UV sanitizing system of claim 10, wherein the perforated separate UV reflector comprises multiple separate perforated UV reflectors.

12. A UV sanitizing system comprising:
a plurality of UV sanitizing modules, wherein each sanitizing module comprises:
one or more UV sensors;
one or more satellite assemblies each comprising a satellite UV illumination source;
a base unit in communication with the one or more UV sensors and the one or more satellite assemblies, the base unit comprising:
one or more base UV illumination sources;
a first memory having stored therein software instructions;
a first processor in communication with the memory and configured to receive the first software instructions that when executed by the first processor cause the base unit to perform operations comprising:
receiving sensor data from the at least one UV sensor, wherein the sensor data is indicative of the amount of UV energy received by the UV sensor;
communicating the UV sensor data to a central control facility;
receiving from the central control facility instructions for controlling at least one of the UV illumination sources of the one or more satellite assemblies and the one or more UV illumination sources of the base station to reduce the UV exposure of the area within the location; and
delivering UV illumination commands to a location in which the one or more satellite assemblies and the base station are present; and
the central control facility comprising:
a second memory having stored therein second software instructions;
a second processor in communication with the second memory and configured to receive the second software instructions that when executed by the second processor cause the central control unit to perform operations comprising:
determining from the sensor data when an area within the location has received sufficient UV energy; and
sending instructions to the base unit for controlling at least one of the UV illumination sources of the one or more satellite assemblies and the one or more UV illumination sources of the base station to reduce the UV exposure of the area within the location when the area within the location has received sufficient UV energy according to the sensor data.

13. The UV sanitizing system of claim 12, wherein the communication between the base unit and the one or more satellite assemblies is over a link selected from the group consisting of a wired connection and a wireless connection.

14. The UV sanitizing system of claim 12, wherein the UV sensor is selected from the group consisting of a portable sensor and a sensor having a fixed location.

15. The UV sanitizing system of claim 14, wherein the fixed location is selected from the group consisting of a fixed location in a room to be sanitized and a piece of patient furniture.

16. The UV sanitizing system of claim 15, wherein the patient furniture is selected from the group consisting of chairs, beds, and operating tables.

17. The UV sanitizing system of claim 12, wherein controlling the at least one of the UV illumination sources of the one or more satellite assemblies and the one or more UV illumination sources of the base station to reduce the UV exposure of the area within the location comprises terminating UV illumination from a selected satellite UV illumination unit when a pre-determined amount of UV illumination is received by the sensor.

18. The UV sanitizing system of claim 12, wherein the location is selected from the group consisting of a doctor's office, a clinic, a patient ward, an operating room, an emergency room, an intensive care unit, a blood donation facility, and a kidney dialysis facility.

19. The UV sanitizing system of claim 12, wherein each sanitizing module further comprises a separate UV reflector.

20. The UV sanitizing system of claim 19, wherein the UV separate reflector comprises multiple separate UV reflectors.

21. The UV sanitizing system of claim 19 wherein the separate UV reflector comprises a perforated UV reflector.

22. The UV sanitizing system of claim 21, wherein the perforated separate UV reflector comprises multiple separate perforated UV reflectors.
